(19) United States
(12) Patent Application Publication
(10) Pub. No.: US 2026/0311866 A1
(43) Pub. Date: Oct. 8, 2026

(54) INCIDENT MONITORING SYSTEMS AND METHODS

(71) Applicant: Voxela, Inc, Fremont, CA (US)

(72) Inventors: Goutham Kamath, Fremont, CA (US); Yuta Endo, Santa Clara, CA (US); Yusuke Yoshida, Tokyo (JP)

(21) Appl. No.: 19/636,659

(22) Filed: Apr. 1, 2026

Related U.S. Application Data
(60) Provisional application No. 63/782,852, filed on Apr. 3, 2025.

(51) Int. Cl. G16H 40/67; G06V 10/70; G06V 20/40; G06V 20/52; G06V 40/10; G06V 40/20; G16H 10/60; G16H 15/00; G06F 40/166

(52) U.S. Cl. CPC G16H 40/67; G06V 10/70; G06V 20/41; G06V 20/44; G06V 20/52; G06V 40/10; G06V 40/25; G16H 10/60; G16H 15/00; G06F 40/166

(57) ABSTRACT

Systems and methods for incident monitoring are described. One aspect includes monitoring a human subject using an imaging device. Data generated by the visual imaging device and associated with the monitoring may be processed to detect a fall of the human subject. A first image sequence and a second image sequence capturing the fall may be recorded. The first image sequence may be temporally shorter than the second image sequence. One aspect includes transmitting an alert associated with the fall along with the first image sequence to a caregiver, receiving metadata associated with the human subject, and generating a report of the fall. The generating may be based on analyzing the second image sequence and the metadata.

### CROSS-REFERENCE TO RELATED APPLICATION

This application claims the priority benefit of provisional patent application No. 63/782,852, titled “Generative AI-Based Incident Report Generation for Elderly Care and Healthcare Environments” filed on Apr. 3, 2025, the disclosure of which is incorporated by reference herein in its entirety.

### BACKGROUND

### Technical Field

The systems and methods described herein relate to monitoring residents such as patients and elderly residents in hospitals, elderly care homes, rehabilitation centers, and assisted living facilities. Some aspects implement incident management systems configured to monitor events and/or incidents such as falls for these residents.

### Background Art

Falls remain one of the most frequent and impactful incidents occurring within healthcare facilities such as nursing homes, assisted living centers, rehabilitation clinics, and hospitals. Such incidents pose substantial health risks, ranging from minor bruises to severe outcomes like fractures, head trauma, or chronic mobility impairment. These incidents place considerable strain on healthcare resources. With aging populations worldwide, the frequency and impact of these fall incidents are anticipated to rise significantly, amplifying clinical challenges and administrative burdens faced by healthcare providers.

Beyond immediate clinical intervention, caregivers are required to meticulously document each fall incident. These manual incident-reporting procedures involve detailed descriptions of the event circumstances, including patient conditions, injuries, contributing environmental factors, and subsequent actions taken. Traditional manual documentation is not only time-consuming but also prone to inconsistencies, inaccuracies, and omissions arising from subjective caregiver interpretations, hurried notes under stressful situations, or fatigue due to workload pressures.

This tedious and subjective documentation task significantly detracts from time caregivers could otherwise devote directly to patient care, potentially affecting the overall quality of care and patient satisfaction.

### Existing solutions and their limitations

### Fall Detection Systems

Current fall detection systems predominantly focus on real-time detection and alert generation using sensors or camera-based AI. These technologies effectively notify caregivers of fall incidents as they occur but typically do not offer any meaningful documentation beyond basic alerts. Caregivers are often left to manually reconstruct detailed event narratives based on limited visual evidence or subjective recall, increasing the risk of incomplete or inaccurate records.

### AI-based Video Analysis Tools

Existing AI-driven video analysis systems can identify and label actions from recorded footage but generally provide context-free textual descriptions. These descriptions lack integration with critical domain-specific metadata, such as room layout, environmental hazards, historical patient data, or prior fall incidents. Consequently, caregivers must still manually contextualize and structure this information into formal incident reports, making the process cumbersome and potentially inconsistent.

Additionally, existing AI-based narrative generation tools found in other sectors, such as security surveillance or sports reporting, typically focus on generic event summarization or captioning and are not optimized for healthcare-specific requirements, such as regulatory compliance or medical accuracy.

### Manual and Semi-automated Documentation

Traditional documentation, even in partially-digitized environments, remains time-consuming, subjective, and error-prone. Caregivers spend considerable effort compiling narratives while risking omissions or inconsistencies due to subjective recall, stress, or fatigue. Moreover, a lack of standardized templates and workflows further complicates incident documentation, particularly when complying with varied regulatory requirements across different jurisdictions or facilities.

### SUMMARY

Aspects of the invention are directed to automated incident report generation for incidents related to residents such as patients and elderly residents.

One aspect includes monitoring a human subject using an imaging device. Data generated by the imaging device may be processed to detect a fall of the human subject. A first image sequence and a second image sequence recoding the fall may be individually recorded. In an aspect, the first image sequence is temporally shorter than the second image sequence. An alert associated with the fall along with the first image sequence may be transmitted to a caregiver. An aspect includes receiving metadata associated with the human subject, and generating a report of the fall. The generating may be based on analyzing the second image sequence and the metadata. The report may be generated in response to a request from the caregiver.

In one aspect, the report may be stored in any combination of an electronic health records (EHR) database an incident database.

In an aspect, the metadata includes any combination of a patient history associated with the human subject, and environment information associated with an environment (e.g., a room) within which the human subject is present.

In an aspect, the patient history includes a fall history associated with the human subject.

In an aspect, the environment information includes a presence of any combination of a wheelchair, furniture, medical apparatus, obstacles, and a liquid spill.

The environment information may include any combination of lighting, door states, window states, and a room temperature.

The detecting of the fall may be performed by a trained artificial intelligence (AI) system. The fall detection may include an estimation of a pose and a gait of the human subject. The pose and gait estimation may include analyzing stability, balance, and movement patterns of the human subject preceding the fall.

In an aspect, the detecting of the fall includes detecting a sudden change in a posture of the human subject, from a standing or a seated posture to a laying down position at a ground level.

In an aspect, the caregiver is able to edit the report. These caregiver edits may be used to update an AI model.

In an aspect, each of the first image sequence and the second image sequence includes visual data of a respective and distinct time period before the fall and a respective and distinct time period after the fall. Each of the first image sequence and the second image sequence may be either of a video image sequence or a still image sequence.

Other aspects may include apparatuses that implement the above method(s).

### BRIEF DESCRIPTION OF THE DRAWINGS

Non-limiting and non-exhaustive embodiments of the present disclosure are described with reference to the following figures, wherein like reference numerals refer to like parts throughout the various figures unless otherwise specified.

FIG. 1 is a block diagram depicting a computer architecture of an incident monitoring system.

FIGS. 2A and 2B are flow diagrams depicting a method to generate an incident report.

FIG. 3 is a block diagram depicting a computer system architecture.

FIG. 4 is a block diagram depicting a computing system.

### DETAILED DESCRIPTION

In the following description, reference is made to the accompanying drawings that form a part thereof, and in which is shown by way of illustration specific exemplary embodiments in which the disclosure may be practiced. These embodiments are described in sufficient detail to enable those skilled in the art to practice the concepts disclosed herein, and it is to be understood that modifications to the various disclosed embodiments may be made, and other embodiments may be utilized, without departing from the scope of the present disclosure. The following detailed description is, therefore, not to be taken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium of expression having computer-usable program code embodied in the medium.

Any combination of one or more computer-usable or computer-readable media may be utilized. For example, a computer-readable medium may include one or more of a portable computer diskette, a hard disk, a random-access memory (RAM) device, a read-only memory (ROM) device, an erasable programmable read-only memory (EPROM or Flash memory) device, a portable compact disc read-only memory (CDROM), an optical storage device, a magnetic storage device, and any other storage medium now known or hereafter discovered. Computer program code for carrying out operations of the present disclosure may be written in any combination of one or more programming languages. Such code may be compiled from source code to computer-readable assembly language or machine code suitable for the device or computer on which the code can be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“SaaS”), Platform as a Service (“PaaS”), and Infrastructure as a Service (“IaaS”)), and deployment models (e.g., private cloud, community cloud, public cloud, and hybrid cloud).

The flow diagrams and block diagrams in the attached figures illustrate the architecture, functionality, and operation of possible implementations of systems, methods, and computer program products according to various embodiments of the present disclosure. In this regard, each block in the flow diagrams or block diagrams may represent a module, segment, or portion of code, which includes one or more executable instructions for implementing the specified logical function(s). It is also noted that each block of the block diagrams and/or flow diagrams, and combinations of blocks in the block diagrams and/or flow diagrams, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flow diagram and/or block diagram block or blocks.

In caregiving environments, there is a need for a system that not only swiftly detects and alerts caregivers of fall incidents but also seamlessly produces structured, comprehensive, and contextually accurate incident reports. Specifically, the industry requires solutions that:

Automate and streamline the detailed reporting workflow, significantly reducing caregiver documentation workload.

Generate consistent, objective, and detailed incident narratives enhanced by integrating environmental metadata, patient history, and action recognition data.

Allow caregivers to easily review, confirm, or minimally edit AI-generated content, thereby shifting their focus from reporting tasks back to direct patient care activities.

Provide customizable reporting templates tailored to specific regulatory or organizational standards, ensuring compliance and ease of adoption across various caregiving settings.

Given the limitations of existing solutions, there is an unmet need for an integrated approach that seamlessly combines real-time fall detection, context-aware AI analysis, and automated, customizable incident reporting. Such a system would empower caregivers to dedicate more attention and resources directly to patient care rather than documentation.

The systems and methods described herein address these gaps by introducing an AI-driven incident summarization platform that provides detailed, structured, and context-rich incident reports. This platform not only captures comprehensive video and metadata surrounding fall incidents but also leverages advanced AI models and customizable templates to produce accurate, regulatory-compliant summaries. Caregivers can then quickly verify, edit if necessary, and submit these AI-generated reports, thereby streamlining administrative processes, significantly reducing errors, and ultimately enabling caregivers to focus more fully on patient-centered care tasks.

The detailed, consistent documentation provided by the systems and methods described herein further supports healthcare facilities in regulatory compliance, enhances incident tracking for preventative analytics, and improves the quality and continuity of patient care across diverse caregiving settings. Moreover, continuous feedback from caregiver interactions with AI-generated reports helps refine the system over time, improving accuracy and efficiency, and elevating overall caregiving standards.

Aspects of the systems and methods described herein are related to incident (e.g., fall) detection at facilities such as hospitals, elderly care homes and rehabilitation centers. Some aspects include advanced AI-driven video analysis, metadata extraction and a customizable template-based report generator to produce standardized and context-rich summaries of fall events.

Automated AI-generated reports generated by the systems and methods described herein significantly streamline incident-reporting workflows by drastically reducing the time caregivers spend manually compiling detailed incident documentation. With the AI system providing comprehensive, structured summaries, including precise details on the incident’s circumstances, environmental context, and prior patient history, caregivers can efficiently review and verify incident reports rather than create them from scratch. This approach allows caregivers to quickly confirm or make minor edits if needed, substantially cutting down on tedious documentation tasks. Consequently, caregivers are empowered to shift their attention back toward direct patient care activities, significantly improving operational efficiency and overall caregiving quality.

Additionally, the detailed reporting capability of the AI systems enhances report consistency and accuracy, thereby supporting even junior caregivers in producing accurate, thorough documentation aligned with professional standards. By incorporating feedback from caregivers through an intuitive editing interface, the system continuously learns and improves, resulting in an ever-increasing accuracy and completeness of incident reports over time. Ultimately, this functionality provides caregiving organizations with a robust and standardized documentation tool, ensuring compliance with regulatory requirements, promoting data-driven risk assessment, and laying the foundation for improved patient safety and preventative care strategies.

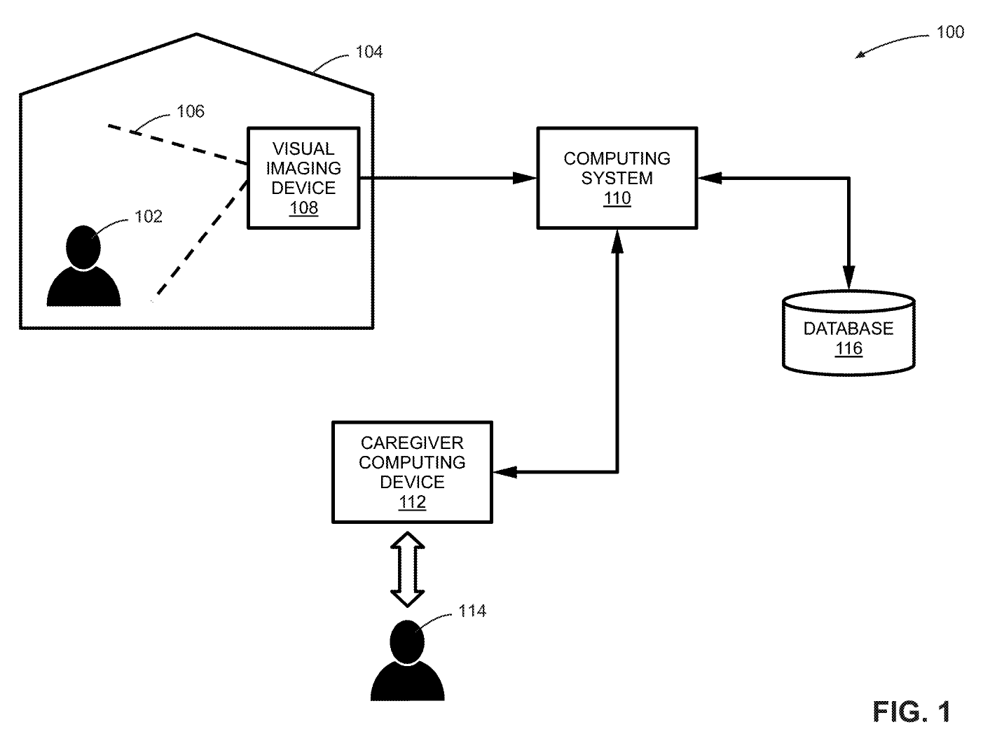

FIG. 1 is a block diagram depicting a computer architecture of an incident monitoring system 100. As depicted, incident monitoring system 100 includes a visual imaging device 108, a computing system 110, a database 116, and a caregiver computing device 112.

In an aspect, visual imaging device 108 monitors a resident 102 in a facility 104. Resident 102 may be a patient or an elderly resident (e.g., a human subject). Facility 104 may be any of a hospital, a nursing home, a rehabilitation center, an elderly care home, or some other care facility.

Each of computing system 110 and caregiver computing device 112 may be based on a computer system architecture that includes, at the very least, a processor, a memory and a network/communication interface. Computing system 110 may be any of a laptop computer, a desktop computer, or a remote server. Embodiments of computing system 110 may be implemented in a cloud computing environment. Caregiver computing device 112 may be any of a laptop computer, a desktop computer, a mobile device (e.g., a mobile phone or a tablet), or any other computing device.

In an aspect, visual imaging device 108 monitors resident 102, where resident 102 is in a field-of-view 106 of visual imaging device 108. In an embodiment, resident 102 is a human subject. Visual imaging device 108 may be any of a video camera, a still image camera, an infrared camera or a depth sensor, or some combination thereof. Visual imaging device 108 may be configured to capture visual data in one or more electromagnetic spectral bands (e.g., infrared, ultraviolet and visual). In an aspect, visual imaging device may include an RGB imaging sensor or a monochromatic imaging sensor. Visual imaging device 108 may be configured to generate any combination of still images and video streams that are transmitted to computing system 110. As presented herein, a video stream (e.g., a sequence of video frames) is considered to be a type of image sequence. Computing system 110 may receive the images and/or video streams, and process the images and/or video streams using one or more trained AI models. These trained AI models may be configured to detect whether an event or incident associated with resident 102 has occurred (e.g., a fall). To perform this event detection, the AI models may perform image processing on the still images and/or video streams, as described herein.

If an incident (e.g., a fall) is detected by computing system 110, then computing system 110 can issue an alert to caregiver computing device 112, along with any relevant captured/recorded images and/or video streams that capture the incident. The alert, the images and/or the video streams may be reviewed by caregiver 114. In response to a confirmation by caregiver 114 that an incident has been detected, caregiver 114 may alert a caregiver team to attend to resident 102, or caregiver 114 may personally attend to resident 102. Caregiver 114 may also request computing system 110 to automatically generate a report of the incident. Computing system 110 may analyze the images and/or video streams using the trained AI model(s), and automatically generate a report (e.g., a text-based report in an appropriate format) that describes the incident. This analysis may include the AI model(s) processing metadata regarding the facility 104. For example, this metadata may include details about an environment (e.g., a room) in which resident 102 is present. The metadata can include information such as a presence of furniture, medical equipment, or a wheelchair, or a presence of a spill. This metadata may be retrieved by computing system 110 from database 116.

In an aspect, the report generated by computing system 110 may be transmitted back to caregiver computing device 112, for review by caregiver 114. The report may also be stored on database 116, as a part of an electronic health record (EHR) or an incident record.

In an embodiment, caregiver 114 may choose to edit the automatically-generated report (e.g., to correct any errors or inconsistencies). These edits may further be ingested by the AI model(s) on computing system 110 to improve future analyses. Such an edited document may be stored on database 116, as a part of the electronic health record (EHR) or incident record.

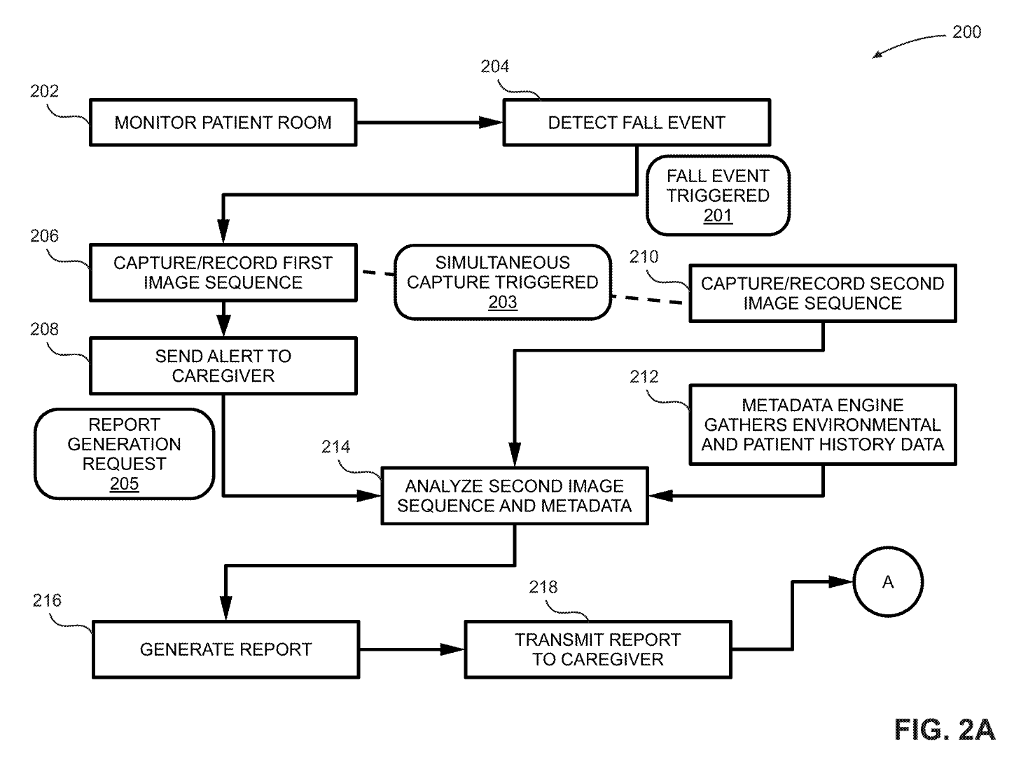

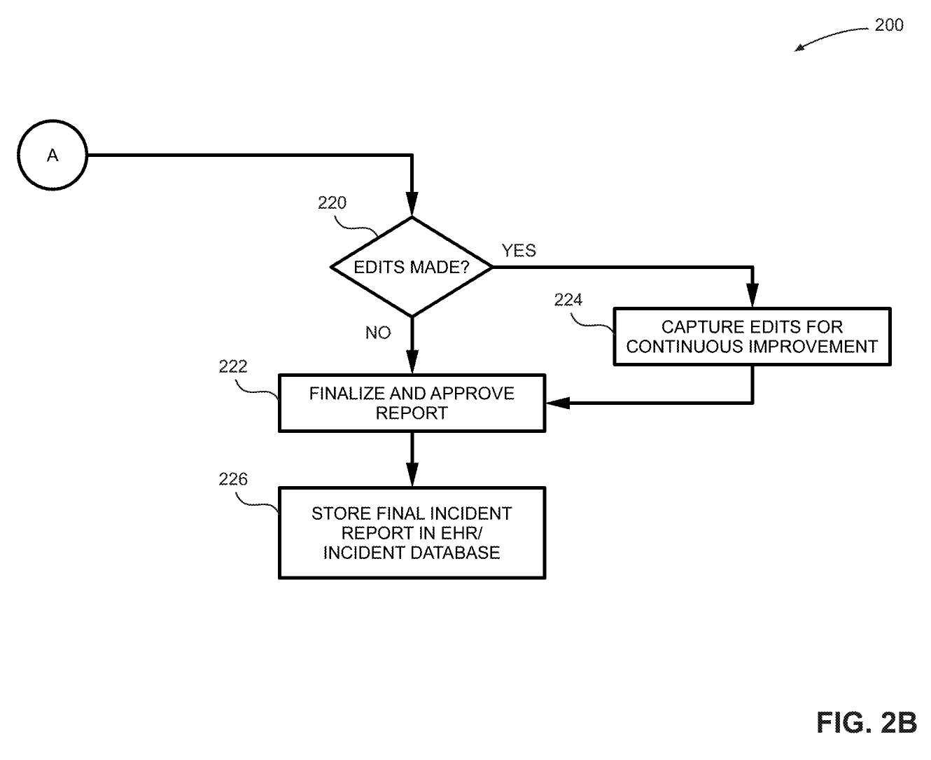

FIGS. 2A and 2B are flow diagrams depicting a method 200 to generate an incident report. Referring to FIG. 2A, method 200 includes monitoring a patient room (202). For example, visual imaging device 108 monitors resident 102 in facility 104. In a particular example, resident 102 is present in a room in facility 104 and in field-of-view 106 of visual imaging device 108. The monitoring 202 may include visual imaging device 108 transmitting an image sequence and/or a video stream to computing device 110. Computing device 110 may process the image sequence and/or the video stream using at least one trained AI model/system to perform a fall detection. In another embodiment, visual monitoring device 108 may be an edge computing device configured to implement one or more AI models to perform operations such as fall detection. The results from fall detection performed on the edge computing component of visual monitoring device 108 may be transmitted to computing system 110 for further analysis.

Method 200 may include detecting a fall event (204). If computing system 110 detects a fall event (i.e., a fall event is triggered 201), then computing system 110 captures or records a first image sequence (or video stream) (206) and a second image sequence (or video stream) (210). In an aspect, the respective captures of the first image sequence (206) and the second image sequence (210) are triggered simultaneously (203).

In an aspect, recording/capturing the first image sequence (206) and the second image sequence (210) includes computing system 110 saving one or more image frames received from visual imaging device 108 in different storage locations. In one aspect, the first captured image sequence/video stream is shorter than the second captured image sequence/video stream. In a particular embodiment, the first captured image sequence/video stream is 30 seconds long, and the second captured image sequence/video stream is 5 minutes long. The first captured image sequence/video stream may include images and/or video from a first time period before the incident, a second time period including the incident, and a third time period after the incident. The second captured image sequence/video stream may include images and/or video from a fourth time period before the incident, a fifth time period including the incident, and a sixth time period after the incident.

Method 200 may include sending an alert to a caregiver (208). In an aspect, the alert may be sent to the caregiver along with the first captured image sequence/video stream. For example, computing system 110 may send the first captured image sequence/video stream to caregiver computing device 112 for review by caregiver 114, along with the alert. Caregiver 114 may review the alert and the first captured image sequence/video stream and send a report generation request (205) to computing system 110 from caregiver computing device 112.

In response to receiving the report generation request (205), method 200 may include analyzing (e.g., by computing system 110) the second captured image sequence/video stream and metadata (214). The metadata may be received from a metadata engine that gathers environmental data and patient history data (212). This metadata may be stored on database 116. The patient history data and second captured image sequence/video stream may be analyzed (214) by computing system 110 to automatically generate a report describing the incident (216). In an alternate embodiment, computing system is configured to analyze the first captured image sequence/video stream in addition to the second captured image sequence/video stream and the metadata and then generate the report. In an aspect, an AI model running on computing system 110 may be configured to generate the report.

Method 200 may include transmitting the report to the caregiver (218). For example, computing system 110 may transmit the report to caregiver computing device 112 for review by caregiver 114.

Referring to FIG. 2B, method 200 may include determining one or more edits have been made to the report by the caregiver (220). For example, when caregiver 114 edits the report on caregiver computing device 112, a copy of the edited report may be transmitted to computing system 110. If computing system 110 determines that caregiver 114 has edited the document, then computing system 110 captures these edits for continuous improvement and training of the AI model (224). For example, edits made by caregiver 114 can be used to inform and train the AI model on computing system 110 to help improve a quality of future reports. After capturing the edits (224), the method goes to 222, where the report is finalized and approved.

If, at 220, the method determines that no edits have been made, the method goes to 222. From 222, the method goes to 226, wherein the finalized incident report is stored in an EHR and/or an incident database (e.g., a finalized version of the report is stored by computing system 110 in database 116).

Details of a particular implementation of incident monitoring system 100 are as follows. The incident monitoring system 100 may be configured as a comprehensive fall detection and reporting system that addresses the shortcomings of existing technologies by automatically creating rich, contextual incident reports. Upon detecting a fall (204) via AI-driven computer vision algorithms (e.g., YOLO, RT-DETR, Florence 2, Qwen 2.5 - VL), the system proceeds with the following steps:

### Video Snippet Capture 206, 210

A critical 30-second clip is immediately recorded (e.g., capturing the first image sequence/video stream at 206) to capture the actual fall event (e.g., a few seconds before and after the detected fall). This short snippet is readily available for quick review, enabling caregivers to gain immediate insight into the incident.

Simultaneously, a longer 5-minute extended video chunk is recorded in the background (e.g., 2.5 minutes prior to the event and 2.5 minutes after) and stored for AI analysis (e.g., capturing the second image sequence/video stream at 210). This longer segment offers additional context leading up to the fall and the immediate aftermath.

### Caregiver Notification and Review 208

The system automatically sends a push notification to designated caregivers (e.g., via a smartphone app, nurse call station, or desktop alert system). For example, computing system alerts caregiver 114 via caregiver computing device 112.

Once tapped, the caregiver is directed to an incident alert page that displays the 30-second highlight clip. This immediate preview ensures quick situational awareness, potentially influencing the caregiver’s next steps (checking vitals, assisting the fallen individual, etc.).

### AI-Driven Summary Generation 212, 214

A “Generate Summary” button on the user interface triggers an AI-based text generation module (e.g., caregiver 114 initiating report generation request 205).

This module processes both the short snippet (e.g., the first captured image sequence/video stream) and the extended 5-minute footage (e.g., the second captured image sequence/video stream), extracting critical details such as sequence of actions, person’s posture transitions, and environmental factors (e.g., furniture placement, lighting conditions).

Metadata from external databases (e.g., database 116) or on-site sensors (room layout, patient history, time of day, etc.) is also fed into the AI pipeline to provide a broader context (for example, a metadata engine gathering environmental and patient history data at 212).

### Structured Report Creation 216

The textual output is automatically inserted into a customizable template, ensuring a standardized format that adheres to facility-specific or regulatory guidelines (e.g., fields such as “Injury Details,” “Environment,” “Potential Cause,” and “Recommendations”).

The result is a comprehensive, context-rich incident report that requires minimal caregiver editing.

### Continuous Improvement and Feedback Loop 220, 224

Caregivers and other medical professionals can edit or annotate the automatically generated report, providing feedback on accuracy or completeness (220).

The system classifies these edits (e.g., minor vs. major) (224) to continuously refine the AI models: metadata extraction, prompt generation, and personalized knowledge for individual patients (e.g., mobility issues or known risk factors).

Through this end-to-end approach, the incident monitoring system 100 reduces documentation workload, improves consistency of incident reports, and offers a scalable framework adaptable to varied healthcare settings.

### Features of incident monitoring system 100 also referred to as “the system”

### Contextual Metadata Input

### Room-Level Metadata

The incident monitoring system 100 can automatically gather and store details such as room size, furniture layout, lighting conditions, and time of day (e.g., in database 116). This input is critical for understanding the environmental context around a fall.

### Object Detection and Action Recognition

Leveraging computer vision models (e.g., YOLO, RT-DETR, Florence 2, Qwen 2.5 - VL) identifies key objects (e.g., chairs, wheelchairs, medical equipment) and the actions of individuals in the room. The AI can detect posture changes from walking or standing to falling, helping to pinpoint the exact cause (e.g., trip, slip, loss of balance). These AI models can be included in computing system 110.

### Pose Estimation and Gait Analysis

Advanced pose-estimation algorithms (e.g., OpenPose, MediaPipe, HRNet) analyze body keypoints to accurately determine an individual’s posture, movement patterns, and balance. By computing metrics such as the individual's centroid and the distance between their feet, the system provides deeper insights into gait stability and postural alignment. This additional metadata enhances the ability of the AI-driven reporting module to precisely identify the underlying causes of falls, such as loss of balance, unstable gait, or missteps, thereby generating more context-rich and informative incident reports.

### Patient/Caregiver History

The system accesses past incident logs, mobility assessments, or known risk factors. For instance, if a patient has previously fallen or uses a walker, that detail is automatically referenced in the final report for clarity and potential risk assessment.

### Template-Based Report Generation

### Customizable and Standardized

Facilities can upload or define their preferred incident report templates—either standard ones (e.g., OSHA guidelines) or user-defined fields. This ensures alignment with local regulations or organizational policies.

### Auto-Population of Key Sections

Fields such as “Injury Details,” “Environment,” “Sequence of Events,” “Possible Causes,” “Follow-Up/Recommendations,” and “Witness Information” are populated from AI-generated text. Metadata (e.g., time stamps, room number) also flows into relevant template fields.

### Seamless Integration

The system can export or sync these structured reports into nurse call systems, EMR platforms, or other clinical documentation software, minimizing repetitive data entry.

### AI Model Enhancement & Prompt Engineering

### Advanced NLP Natural Language Processing

Harnessing next-generation models (e.g., GPT-4o, Gemini 1.5 Flash) to interpret video analysis outputs and metadata, converting them into coherent paragraphs with attention to clarity and correctness.

### Context-Aware Prompts

The text generation process is guided by carefully engineered prompts that incorporate relevant context (e.g., recognized actions, environment data, patient history, etc.). This ensures that the final summary is accurate, domain-specific, and reflects the event’s causal factors.

### Continuous Improvement

### Feedback Capture

Every time a caregiver or medical professional edits an AI-generated report, the system logs these changes as structured feedback.

### Adaptive Learning

Based on the nature of edits (e.g., removing irrelevant details, correcting inaccurate statements, adding missing context, etc.), the system updates its metadata extraction approach, prompt design, or stores facility-specific memory for future incident summaries.

### Personalization for Residents

The system can also retain specific user or patient preferences. For instance, if a certain patient regularly uses a walker, the AI ensures future reports automatically reference or check for walker presence, or note potential hazards related to it.

### Privacy and Data Security

One aspect of the invention includes compliance with data protection regulations (e.g., HIPAA) by encrypting video streams, anonymizing sensitive data in textual summaries (where legally necessary), and offering access controls to authorized staff only.

Video footage can be deleted or archived in compliance with local regulations once it has served its purpose in the reporting process.

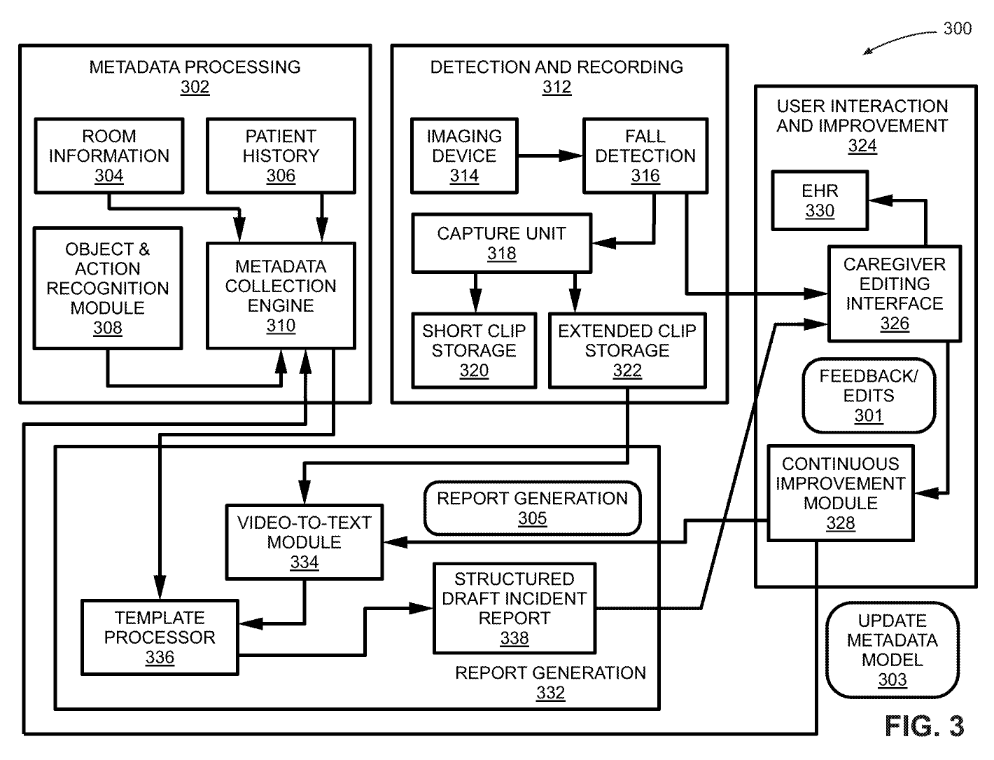

FIG. 3 is a block diagram depicting a computer system architecture 300. As depicted, computer architecture 300 includes a metadata processing module 302, a detection and recording module 312, a user interaction and improvement module 324, and a report generation module 332. Metadata processing module 302 further includes room information 304, a patient history 306, an object action and recognition module 308, and a metadata collection engine 310. Detection and recoding module 312 further includes an imaging device 314, a fall detection module 316, a capture unit 318, a short clip storage 320, and an extended clip storage 322. User interaction and improvement module 324 further includes a caregiver editing interface 326, one or more electronic health records (EHR) 330, and a continuous improvement module 328. Report generation module 332 further includes a video-to-text module 334, a template processor 336, and a structured draft incident report 338. Computer system architecture 300 may be implemented at least in part on computing system 110.

In an aspect, metadata collection engine 310 receives inputs from room information 304, patient history 306, and object and action recognition module 308.

Room information 304 may include the following room-level metadata:

Dimensions and Layout: Basic information about the room’s size, furniture arrangement, and obstacles (e.g., chairs, tables, medical devices).

Environmental Conditions: Lighting (ambient vs. overhead), door/window states, temperature, or any special hazards (wet floor signs, cables, etc.).

Patient history 306 may include the following occupant/resident data and past history:

The metadata collection engine 310 can reference health records or incident logs indicating a resident’s previous falls, known mobility issues, or caregiver notes (e.g., “requires walker,” “vision impaired,” “fall risk level”).

This information may be stored in a relational database or a secure healthcare information system (not shown in FIG. 3), ensuring that personal health data is accessed only as needed for the incident summary.

In an aspect, metadata processing module 302 implements metadata retention and versioning, where metadata used in the final report is versioned alongside the captured image sequence(s)/video stream(s) to maintain an audit trail. If the environment or patient data changes later, the original record associated with the incident remains intact for compliance and legal traceability.

In an aspect, imaging device 314 is similar to visual imaging device 108. An image sequence and/or video stream output by imaging device 314 may be analyzed by fall detection module 316, to detect a fall. If a fall is detected, fall detection module 316 triggers capture unit 318 to store a short clip (i.e., short clip storage 320) corresponding to the first captured image sequence/video stream, and an extended clip (i.e., extended clip storage 322), corresponding to the second captured image sequence/video stream.

If a fall is detected, fall detection 316 may send an alert to caregiver 114 via caregiver editing interface 326. The stored short clip may be sent to caregiver computing device 112 from short clip storage 320. If caregiver 114 requests a report via continuous improvement module 328 (e.g., report generation 305), then the second captured image sequence/video stream is transmitted to video-to-text module 334 from extended clip storage 322. Video-to-text module 334 may generate a textual description of the incidents in the video, and then transmit the textual description to template processor 336. Template processor 336 receives the textual description from video-to-text module 334, along with metadata from metadata collection engine 310, to generate structured draft incident report 338. Structured draft incident report 338 is transmitted to caregiver editing interface 326 for review by caregiver 114.

Caregiver 114 may choose to edit the report; this feedback and/or edits 301 is/are transmitted back to video-to-text module 334 via continuous improvement module 328. These updates are used to update the structured draft incident report 338. These updates may also be used to update the metadata model (303).

In an aspect, the detection and recording module 312 implements a sensing/detection module that includes the following:

### Camera-Based System

Detection and recording module uses one or more wall-mounted or ceiling-mounted cameras (e.g., imaging device 314) to observe the environment. These can be standard RGB cameras with IR sensors for night time vision. These cameras can be IP based cameras, depending on the facility’s requirements.

AI Models (e.g., YOLO, RT-DETR, Florence 2, Qwen 2.5 - VL) are applied either locally on an edge device (with GPU capabilities) or on a server (e.g., computing system 110) to detect suspicious postural changes indicative of a fall.

### Detection Criteria

Real-time action recognition or pose estimation is performed. If the algorithm identifies an abrupt change from a vertical stance to lying on the floor, or detects inactivity at ground level beyond a threshold time, the system flags a fall event. This analysis may be performed by fall detection module 316.

A fall event is passed into several other models (that may be included in fall detection module 316) using ensemble techniques and finally, the result is aggregated. This process has been found this to improve the accuracy of detecting a fall.

### Event Trigger

Once a fall is detected, the system marks the start time and triggers subsequent modules to initiate video capture and metadata processing.

In an aspect, the detection and recording module 312 implements a video capture and storage capability, with the following features:

### Automatic Clip Saving

Immediately after a fall detection, a 30-second highlight clip is saved to local or cloud storage (e.g., the first captured image sequence/video stream, saved to short clip storage 320). The system includes a buffer that retains a few seconds of video prior to the detection (e.g., a ring buffer) to ensure that the critical moment leading up to the fall is preserved.

This short clip serves for quick review by caregivers, enabling fast understanding of how the fall occurred.

### Extended Context Clip

In parallel, the system extracts a longer 5-minute chunk (e.g., 2.5 minutes pre-fall and 2.5 minutes post-fall) (e.g., the second captured image sequence/video stream, saved to extended clip storage 322). This extended context is particularly important for AI analysis (e.g., identifying pre-fall conditions or immediate aftermath).

This longer video segment is stored separately and may be secured in an encrypted format to comply with data privacy regulations (e.g., HIPAA) and to limit access only to authorized personnel.

### Video Storage & Retrieval

Both video clips are indexed in a database (that may include short clip storage 320 and extended clip storage 322) with a unique incident ID, tied to resident data (if available) and any relevant metadata (date, time, location). This structure facilitates easy retrieval during or after the report generation process.

In an aspect, any or both of object and action recognition module 308 and fall detection module 316 include an action recognition engine, that implements the following:

### AI-Based Human Action Detection

Uses deep neural networks to identify actions such as sitting, standing, walking, reaching, or collapsing. This component is crucial for pinpointing the sequence of events leading to a fall.

Each detected action is timestamped and assigned a confidence score.

### Object Detection

In addition to human pose or action, the system identifies relevant objects (e.g., tables, wheelchairs, medical apparatus, spilled liquid).

By correlating object positions with human actions, the AI can determine potential causal factors (e.g., tripping over a chair leg, slipping on a wet floor).

### Pose and Gait Estimation

Advanced pose-estimation algorithms (e.g., OpenPose, MediaPipe, HRNet) are used to detect precise human body keypoints and joint positions.

The system computes essential biomechanical metrics, such as the individual’s body centroid, distance between feet, and stride length, enabling detailed gait analysis.

This detailed information provides insights into stability, balance, and movement patterns preceding a fall, significantly enriching the contextual understanding of fall events.

### Additional Context for Reporting

The recognized actions and objects feed into an event timeline that the AI references during report generation.

This timeline may reveal key triggers, such as a sudden step forward, an attempt to grasp a support rail, or an obstacle left on the floor.

In an aspect, report generation module 332 includes the following:

### AI-Based Video-to-Text video-to-text module 334

After processing the 5-minute context clip, a video summarization algorithm (e.g., a custom Qwen 2.5 VL or Gemini 1.5 Flash model adapted for video data) synthesizes the major events into a concise narrative. This aspect may be implemented by video-to-text module 334.

The narrative focuses on brief incident description, type of fall (e.g., loss of balance, falling during transition), environmental conditions, head impact detection, post-fall analysis, follow-up-plan

### Template Processor 336

The template processor 336 inserts the generated text into a customizable incident report template. Each facility may define unique sections such as “Environmental Conditions,” “Observations,” “Injuries Noted,” “Primary Cause,” “Follow-Up Care,” etc.

Metadata (e.g., room number, date/time, relevant past falls) is automatically populated into the respective fields, ensuring factual completeness and standardized formatting. This metadata may be received by template processor 336 from metadata collection engine 310.

### Integration with Facility Systems

Completed reports can be exported (e.g., PDF, XML, or HL7 format) or synchronized with electronic health record (EHR) systems or nurse call systems for further action.

If configured, automatic notifications can be sent to relevant stakeholders (e.g., the resident’s physician, nurse manager, or risk management team).

An example methodology implemented by incident monitoring system 100 is presented.

### Video Analysis

### Feature Extraction

The system employs convolutional neural networks (CNNs) or transformers specialized for video frames to identify persons, actions, and key environmental elements.

Temporal coherence is established by analyzing frame sequences, detecting unusual acceleration or posture shifts characteristic of a fall.

### Person and Hazard Identification

If multiple individuals are in the frame, the AI differentiates them, labeling the primary subject who fell versus bystanders or caregivers.

Potential hazards (wet floors, obstacles, loose cables) are flagged for mention in the final summary.

### Multi-Modal Data Fusion

Data from depth sensors (if used), time-of-day logs, and occupant history are combined to enrich the AI’s understanding of the incident context.

### Action Recognition, Pose and Gait Estimation

### Transition Detection

The engine tracks transitions (e.g., standing to lying) with frame-by-frame analysis, assigning timepoints to each major change.

Causality or trigger identification (e.g., tripping vs. fainting) is inferred by analyzing how quickly the posture changes and what objects are involved.

### Group Interaction (Optional)

If more than one person is present (e.g., a caregiver assisting a resident), the AI can note interactive actions (attempted catch, assistance, etc.).

### Metadata Integration

### Data Aggregation

The system collects lighting conditions, room size, occupant info, and any historical references to past falls involving the same person or location.

This metadata is fed into the prompt for AI-based text generation, shaping how the final summary is written (e.g., “This is the second fall in three months for Resident X…”).

### Tailored Output

By using metadata-driven prompt templates, the AI ensures the summary references specifically relevant details rather than generic descriptors (e.g., “The resident was found near the bed in the northwest corner of the room, which had poor lighting conditions at the time.”).

### Quality Control & Editing

### Human-in-the-Loop Review

The system generates a draft report that caregivers can review via a user-friendly interface.

Caregivers have the option to add or modify any incorrect or missing details (e.g., injury specifics, observed behaviors not captured on camera).

### Feedback Incorporation

Edits are tracked and classified (minor vs. major).

Major edits may trigger updates to prompt generation or to the metadata extraction approach. Minor edits might be stored as user-specific preferences (e.g., style guidelines for writing the final report).

Over time, this iterative feedback loop refines the system’s accuracy and efficiency.

An example workflow for implemented by incident monitoring system 100 is presented.

### 1. Fall Detection

The system’s Sensing/Detection Module notices a sudden change in posture (e.g., from standing to lying).

### 2. Video Recording

A 30-second highlight clip is saved for immediate review; a 5-minute extended recording is stored for AI analysis.

### 3. Notification

Push alerts are sent to caregiver devices.

### 4. Immediate Incident View

Caregiver taps the alert, sees the 30-second clip to confirm the fall and check the individual’s status.

### 5. Generate Summary

Caregiver selects “Generate Summary,” which triggers the AI-based summarization of the extended clip plus any relevant metadata.

### 6. AI Draft and Editing

A structured incident report is auto-filled with text describing the sequence of events, environmental context, and potential root causes.

Caregiver reviews and makes minor corrections or annotations.

### 7. Finalization & Storage

The final report is saved in the system, optionally pushed to EHR or nurse call systems, and archived along with its associated video clips.

Other features that can be included in incident monitoring system 100 are described.

### 1. Integration with Electronic Health Records (EHR)

The system can auto-populate patient details and retrieve prior fall incidents directly from EHRs to include in the generated report.

Completed reports can be stored back into the patient’s medical record in real-time.

### 2. Multilingual Template Support

Facilities operating in multilingual regions can implement language models with localized templates, enabling the system to generate summaries in multiple languages, catering to diverse caregiver teams.

### 3. Advanced Privacy and Data Security

Encryption at rest and in transit ensures HIPAA/GDPR compliance.

Access controls limit which staff members can view or edit specific incidents.

Automated deletion schedules or retention policies can be set for video data to meet local regulatory requirements.

### 4. Mobile and Wearable Integration

The invention can augment data from mobile devices, smartwatches, or wearable sensors that detect sudden movement or impact. This multi-modal approach increases fall detection accuracy and provides a more robust incident context.

### 5. Continuous Model Training

In some configurations, the system may periodically retrain its AI models using anonymized data, improving fall detection and report generation capabilities over time.

### 6. Analytics and Prevention

Historical incident data can be analyzed for patterns (e.g., repeat falls in a specific room or time of day). The system may recommend preventative measures such as rearranging furniture, improving lighting, or scheduling additional check-ins.

### 7. Continuous Care Logging and Auditing

The system’s automated reporting capabilities can be expanded beyond fall incidents to enable comprehensive, continuous logging and auditing of caregiver activities.

By leveraging AI-driven monitoring, the invention can objectively track, summarize, and validate caregiving activities, automatically updating care logs with timestamped evidence (video or image clips) for auditing purposes.

This capability ensures accurate caregiver activity documentation, promotes transparency, prevents potential fraud or abuse of incentive programs (particularly those funded by federal grants or government agencies), and enhances overall compliance and quality of care standards.

### 8. Remote Physiotherapy (PT) Resident Movement Monitoring and Reporting

Certain fall incidents can be escalated automatically for remote review by Physiotherapists (PT).

Physiotherapists can log into the system remotely, view recorded incident videos, resident movement patterns, pose estimation metrics, gait analysis, and other relevant video clips associated with the resident.

This capability enables physiotherapists to remotely assess incidents, accurately evaluate resident mobility, identify potential risks, and provide informed, timely recommendations, thereby supporting proactive care management and enhancing rehabilitation outcomes.

### Example use case #1: Hospital Room

### Setting & Environment

A hospital room equipped with ceiling-mounted cameras monitoring patient activity.

The room layout is complex, with a bed, IV stand, side table, and other medical devices that may contribute to trip hazards.

Lighting conditions vary by day/night shifts—late-night dim lights vs. daytime brightness.

### Fall Detection Trigger

The patient attempts to stand from the bed without support around 2 a.m. The AI-based detection module notices a sudden posture change from partially seated to lying prone on the floor.

A fall event is flagged with high confidence.

### Video Capture and Storage

Highlight Clip: A 30-second snippet (e.g., 15 seconds before and 15 seconds after the detected fall) is saved for quick caregiver review.

Extended Clip: A 5-minute segment (2.5 minutes prior, 2.5 minutes after) is recorded to capture context (e.g., whether the patient was restless, attempting to reach for something, or if the room lighting was too low).

### Metadata Collection

Room Metadata: The system records the bed position, local time (2:02 a.m.), and lighting conditions (low ambient light).

Patient Mobility Records: The patient’s EMR indicates previous difficulty standing unaided due to recent surgery. The system automatically pulls this detail from the hospital’s EHR.

### Action Recognition and Object Detection

The AI identifies the patient’s attempt to stand and the subsequent slip or loss of balance.

Objects in close proximity (e.g., the IV stand) are noted. The system checks if the patient tried to use it for support.

### Report Generation

Draft Summary: AI constructs a narrative: “At approximately 2:02 a.m. in Room 304, Patient X attempted to stand without support and lost balance near the bed corner. The lighting level was low, and the patient has a known mobility challenge post-surgery.”

Template Integration: The text is placed into the hospital’s Incident Report template under headings like Event Summary, Environment Details, Patient Condition, Injury (if any), Recommendations.

A recommended follow-up is auto-suggested (e.g., “Assess viability of installing bed rails or nighttime support devices”).

### Caregiver Review and Finalization

A caregiver reviews the auto-generated content, confirms the details, and adds any immediate observations about the patient’s injuries.

The final report is then stored in the hospital’s incident log and linked to the patient’s chart in the EHR.

### Outcome

The system minimizes the time staff spend on documentation while ensuring consistent, data-rich incident reporting. The detailed context (time, lighting, bed position, patient history) aids in root-cause analysis and may lead to preventive measures, such as providing an assistive device.

### Example use case #2: Nursing Home Common Area

### Setting and Environment

A large common area in a nursing home where multiple residents gather for activities and social interaction. Overhead cameras are strategically placed to minimize blind spots.

Furniture items—chairs, sofas, tables—are scattered around, and the floor surface can sometimes be slippery if spills occur.

### Fall Detection Trigger

During a busy afternoon, the AI detects an abrupt posture change of a resident near a table. The system recognizes a fall from walking upright to lying on the floor.

### Video Capture and Storage

Highlight Clip: A 30-second video highlights the slip moment, including a few seconds prior to the incident.

Extended Clip: 5 minutes of footage (2.5 minutes before, 2.5 after) is stored for further analysis, potentially revealing environmental conditions or interactions with other residents.

### Metadata Collection

Real-Time Object Recognition: The system notes multiple chairs, a table, and a visible wet patch on the floor from a spilled drink.

Occupant Data: The resident’s history shows a previous fall in the same common area, albeit a year ago. The memory module references this for context.

### Action Recognition and Object Detection

The system pinpoints a slipping action in close proximity to the wet patch, suggesting a possible causal link.

It also identifies bystanders or caregivers who might have moved to assist the resident.

### Report Generation

Draft Summary: “Resident Y was observed slipping on a wet floor near the center table in the common area at 3:15 p.m. The system detected a spill on the floor surface, which likely contributed to the fall. This resident experienced a similar fall in the same location last year.”

Template Integration: The text is inserted into a nursing home incident report template, automatically filling sections like Environmental Hazards, Potential Cause, Recommendations.

An automated recommendation appears: “Wipe spill immediately, place signage indicating slippery floor, and review procedures for promptly addressing spills.”

### Caregiver Review and Follow-up

Staff confirm the slip was indeed due to the spill, and a note is added that the resident did not sustain major injuries but was taken for a check-up.

The system flags a pattern of repeated slips in this area, possibly prompting management to rearrange furniture or institute stricter spill-handling protocols.

### Outcome

The AI’s object recognition and action detection layers reveal a preventable cause (wet floor). Automated recommendations in the report guide staff to take immediate corrective measures and consider long-term improvements. The comprehensive summary, with minimal caregiver intervention, ensures consistent incident tracking and demonstrates compliance with safety regulations.

### Additional Applications of the Incident Monitoring System 101

### Assisted Living Private Apartments

Each resident’s private apartment is equipped with discreet, privacy-focused cameras. A fall detection alert triggers the same process: 30-second snippet + extended 5-minute video, metadata (lighting, furniture arrangement), and a structured incident report.

Voice Integration: Some implementations include voice-activated prompts, where the resident (if able) verbally confirms the fall, adding further context to the report.

### In-Home Care for High-Risk Individuals

Similar systems can be deployed in personal residences for individuals at high risk of falls (e.g., post-operative patients). The invention’s remote reporting capability allows families or remote caregivers to receive push notifications and auto-generated incident reports, thereby improving care coordination.

### Rehabilitation Centers

Rehab facilities often require documenting falls during physical therapy sessions. The system integrates data from wearable sensors (e.g., pedometers, motion trackers) to analyze why the patient fell during a specific exercise.

The final report may note therapeutic exercises in progress, cautioning the therapy staff about necessary modifications or additional support.

### Integration with Telehealth Platforms

A future embodiment might allow telehealth providers to instantly view incident footage and read the AI-generated report, facilitating virtual consultations about the resident’s condition and next steps.

### Advanced Analytics and Prevention

Collected data from multiple falls can feed into facility-wide analytics, identifying trends or high-risk zones within a building. This helps administrators implement targeted interventions such as improved signage, floor treatments, or training for staff on proactive fall prevention.

### Continuous activity logging and Textual Search

The incident monitoring system 100 can further support continuous activity logging by automatically recording and summarizing caregiver-resident interactions through AI-based action recognition. These activity logs, enriched with textual metadata, enable efficient auditing, compliance verification, fraud prevention, and quick textual searches, significantly improving operational transparency and caregiver accountability.

One aspect includes A system for automated fall incident reporting, that includes:

A detection module configured to identify a fall event by analyzing at least one video stream or sensor input to detect a change in posture indicative of a fall.

A video capture unit configured to record (i) a highlight clip corresponding to a time interval around the detected fall event and (ii) an extended context clip to provide additional pre- and post-fall information.

A metadata collection component configured to gather environmental data (including room layout, lighting conditions, and occupant information) and historical fall data (including prior incidents or risk factors).

A report generation module employing an AI-based text generation algorithm that integrates video analysis outputs with the gathered metadata to produce a structured incident report in a template-defined format.

A continuous improvement module configured to collect caregiver feedback and edits made to generate reports, classify the edits, and utilize this feedback to iteratively enhance the accuracy and relevance of subsequent AI-generated incident reports.

In an aspect, the report generation module further comprises an object detection sub-module to identify furniture, medical equipment, or other items in the environment that may be relevant to the fall incident.

In an aspect, the metadata collection component captures lighting conditions, time of day, and historical incident records for a specific individual or location, and automatically includes such data in the incident report.

In an aspect, the system includes an editing interface accessible by authorized personnel (e.g., caregiver 114), the interface allowing a user to review and refine the AI-generated incident report before final submission.

In an aspect, the structured template for the incident report is customizable based on facility requirements or provided by an external party, ensuring compliance with regulatory or organizational standards.

In an aspect, the detection module utilizes machine learning algorithms selected from the group consisting of YOLO, RT-DETR, Florence 2, Qwen 2.5 - VL, or equivalent computer vision models to identify a fall event.

In an aspect, the system is configured to integrate with an electronic health record (EHR) system, enabling automatic retrieval of patient data and storing of completed incident reports in the patient’s record.

In an aspect, the report generation module includes a feedback capture mechanism that updates the AI-based text generation algorithm based on user edits, thereby improving future incident summaries.

In an aspect, the extended context clip is at least five minutes in duration, captures pre- and post-fall activities to facilitate enhanced root-cause analysis and contextual reporting.

In an aspect, the detection module is configured to use real-time alerts via a mobile application or nurse call system, such that caregivers are immediately notified to review the highlight clip.

A method of generating an automated incident report for a fall event may include:

* detecting the fall event by analyzing a video feed or sensor input and identifying a sudden change in posture;

* capturing a short highlight clip and a longer extended clip around the detected fall event, the highlight clip focusing on the moment of the fall and the extended clip providing additional context

* collecting contextual metadata relevant to the environment and the individual involved in the fall, including at least lighting conditions, room layout, and historical fall incidents;

* analyzing the captured video to extract key actions and objects, including any furniture, medical equipment, or environmental hazards; and

* incorporating the analysis results and metadata into a structured incident report template via a text generation algorithm, thereby producing an automated fall incident summary.

One aspect includes prompting a caregiver to review the automatically generated report, wherein user edits or annotations are recorded to improve future AI-generated summaries.

In an aspect, the step of detecting the fall event is performed using deep learning-based action recognition models that identify a transition from standing or seated posture to a prone position on the floor.

In an aspect, the structured incident report is exported to an external system selected from the group consisting of electronic medical record (EMR) systems, nurse call platforms, or risk management software.

One aspect includes encrypting the video clips and metadata to ensure data privacy and compliance with healthcare regulations prior to storing or transmitting the incident report.

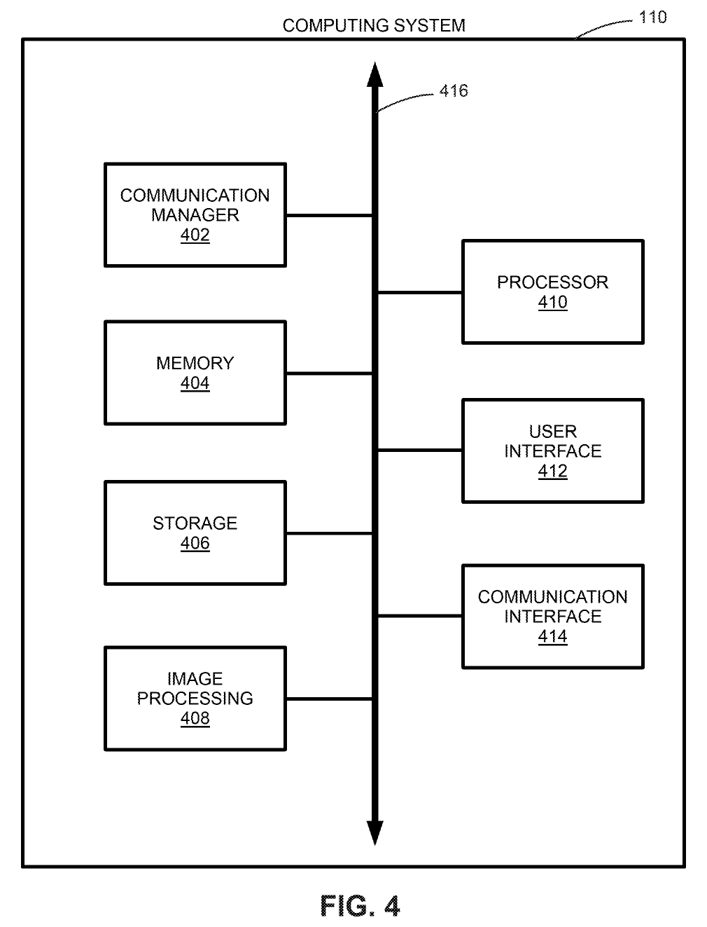

FIG. 4 is a block diagram depicting computing system 110. As depicted, computing system 110 includes a communication manager 402, a memory 404, a storage 406, image processing 408, a processor 410, a user interface 412, a communication interface 414, and a bus 416.

In some embodiments, communication manager 402 is configured to manage communication protocols and associated communication with external peripheral devices as well as communication with other components in computing system 110. Memory 404 may be configured to store data associated with computing system 110. In one aspect, memory 404 includes both long-term memory and short-term memory. Memory 404 may be comprised of any combination of hard disk drives, flash memory, random access memory, read-only memory, solid state drives, and other memory components.

In an aspect, memory 404 includes a non-transitory computer medium. Memory 404 may be comprised of any combination of volatile and non-volatile memory components. Examples of components that may be used to implement memory 404 include random-access memory (RAM), read-only memory (ROM), electrically-erasable programmable read-only memory (EEPROM), flash memory, magnetic memory, optical memory, and so on. Memory 404 may include machine-readable instructions that may be executable by a processor such as processor 410. These machine-readable instructions when executed by the processor 410 cause the processor 410 to perform one or more method steps of an embodiment described herein.

Computing system 110 may include storage 406, that further includes one or more long-term storage devices such as hard disk drives, magnetic drives, magnetic tape, optical storage media (e.g., compact disks (CDs) or digital versatile disks (DVDs)), and so on. Storage 406 may be implemented as a non-transitory computer-readable medium. Storage 406 may be configured to store data and/or instructions related to the operation of computing system 110.

In an aspect, image processing 408 is configured to implement the image and/or video processing aspects of incident monitoring system 100. Image processing 408 may be implemented by any combination of general-purpose processors, or dedicated processing components such as graphics processing units (GPUs).

A processor 410 included in some embodiments of computing system 110 is configured to perform functions that may include generalized processing functions, arithmetic functions, and so on. Processor 410 is configured to process information associated with the systems and methods described herein. Processor 410 may be configured as any combination of microcontrollers, microprocessors, digital signal processors (DSPs), field-programmable gate arrays (FPGAs), graphics processing units (GPUs), accelerated processing units (APUs), central processing units (CPUs), application-specific integrated circuits (ASICs), and so on. Processor 410 may be embodied as a single-core processor, or a multi-core processor. Processor 410 may be implemented as a centralized processor, or in a distributed manner (e.g., a distributed computing system).

User interface 412 allows a user to interact with embodiments of the systems described herein. User interface 412 may include any combination of user interface devices such as a keyboard, a mouse, a trackball, one or more visual display monitors, touch screens, incandescent lamps, LED lamps, audio speakers, buzzers, microphones, push buttons, toggle switches, and so on.

Communication interface 414 may be used to interface computing system 110 with other computing devices and/or computer networks. Examples of computer networks include a local area network (LAN), a wide area network (WAN), the Internet, and so on. Communication interface 414 may support any combination of wired and wireless connectivity/communication protocols such as Ethernet, Wi-Fi, Bluetooth, ZigBee, etc. may be used to interface computing system 110 with other computing devices and/or computer networks. Examples of computer networks include a local area network (LAN), a wide area network (WAN), the Internet, and so on. Communication interface 414 may support any combination of wired and wireless connectivity/communication protocols such as Ethernet, Wi-Fi, Bluetooth, ZigBee, etc. Bus 416 communicatively couples and allows for communication between the different components of computing system 110.

### Additional Embodiments

In some embodiments, contextual visual data associated with a detected fall event may be captured using a rolling context window or dynamic visual buffer, or some other visual data acquisition workflow/algorithm, rather than two separately stored video clips/image sequences.

In some embodiments, a report may be generated from one or more contextual visual data windows, including any combination of continuous video, buffered video, sampled frames, still images, or keyframes associated with the event.

In some embodiments, the techniques described herein for fall-event reporting may also be adapted for other resident safety or caregiving-related events, including bed-exit events, near-fall events, loss-of-balance events, unsafe transfer events, prolonged floor presence, nighttime wandering, or abnormal inactivity. The systems and methods described herein can thus be used for monitoring a broad range of incidents associated with a resident.

In some embodiments, the system may generate structured caregiver documentation, including incident reports, care notes, audit log entries, escalation summaries, or other structured clinical or operational records using customizable templates. In other words, the systems and methods described herein can be configured to generate multimodal, AI-based documentation based on visual data and metadata inputs.

In some embodiments, report generation may be based on visual data together with one or more additional data sources, including depth sensors, infrared sensors, audio inputs, wearable devices, bed sensors, motion sensors, nurse call systems, electronic health record data, and caregiver-entered notes. In other words, a sensor suite with different sensing modalities can be used as a source of different input data sources to the incident monitoring system 100. These different inputs can be appropriately processed (e.g., by any combination of computing system 110 and an edge device processing functionality on a relevant sensing device). In an aspect, a data fusion workflow may be used to aggregate the data from the different sensing sources. This aggregated data may then be used to perform the data processing associated with the incident monitoring workflows described herein.

In some embodiments, the system may reconstruct a temporal sequence of actions before, during, and after a detected event, and may identify causal or contributing factors associated with the event.

In some embodiments, caregiver edits, approvals, or corrections may be used to refine prompts, templates, metadata interpretation rules, or AI model behavior, including facility-specific or workflow-specific adaptation.

In some embodiments, generated documentation may be routed for review, approval, escalation, synchronization, archival, or audit processing within one or more facility systems.

In some embodiments, the system may be used for continuous care logging and creation of audit-ready care records supported by timestamped visual evidence, contextual metadata, and generated summaries.

In some embodiments, one or more portions of the workflow may be performed on a camera device, edge device, gateway, server, cloud platform, or hybrid computing architecture, or any combination thereof.

Although the present disclosure is described in terms of certain example embodiments, other embodiments will be apparent to those of ordinary skill in the art, given the benefit of this disclosure, including embodiments that do not provide all of the benefits and features set forth herein, which are also within the scope of this disclosure. It is to be understood that other embodiments may be utilized, without departing from the scope of the present disclosure.

What is claimed is:

1. A method comprising:
monitoring a human subject using an imaging device;
detecting a fall of the human subject based on processing data generated by the imaging device and associated with the monitoring;
recording a first image sequence capturing the fall and a second image sequence capturing the fall, wherein the first image sequence is temporally shorter than the second image sequence;
transmitting an alert associated with the fall along with the first image sequence to a caregiver;
receiving metadata associated with the human subject; and
generating a report of the fall, wherein the generating is based on analyzing the second image sequence and the metadata.

2. The method of claim 1, further comprising storing the report in any combination of an electronic health records (EHR) database and an incident database.

3. The method of claim 1, wherein the metadata includes any combination of a patient history associated with the human subject, and environment information associated with an environment within which the human subject is present.

4. The method of claim 3, wherein the patient history includes a fall history associated with the human subject.

5. The method of claim 3, wherein the environment information includes a presence of any combination of a wheelchair, furniture, medical apparatus, obstacles, and a liquid spill.

6. The method of claim 3, wherein the environment information includes any combination of lighting, door states, window states, and a room temperature.

7. The method of claim 1, wherein the detecting of the fall is performed by at least one trained artificial intelligence (AI) system.

8. The method of claim 1, wherein the detecting of the fall further comprises performing an estimation of a pose and a gait of the human subject.

9. The method of claim 8, wherein the pose and gait estimation further comprises analyzing stability, balance, and movement patterns of the human subject preceding the fall.

10. The method of claim 1, wherein the detecting of the fall further comprises detecting a sudden change in a posture of the human subject, from a standing or a seated posture to a laying down position at a ground level.

11. The method of claim 1, wherein the caregiver is able to edit the report.

12. The method of claim 11, wherein an AI system uses the caregiver edits to update an AI model.

13. The method of claim 1, wherein the report is generated responsive to a request from the caregiver.

14. The method of claim 1, wherein each of the first image sequence and the second image sequence includes visual data of a respective and distinct time period before the fall and a respective and distinct time period after the fall.

15. The method of claim 1, wherein each of the first image sequence and the second image sequence is either of a video image sequence or a still image sequence.

16. A system comprising:
an imaging device configured to perform a visual monitoring of a human subject; and
a computing system communicatively coupled to the imaging device, wherein the computing system is configured to:
receive data associated with the visual monitoring;
detect a fall of the human subject based on processing the data;
record a first image sequence capturing the fall and a second image sequence capturing the fall, wherein the first image sequence is temporally shorter than the second image sequence;
transmit an alert associated with the fall along with the first image sequence to a caregiver;
receive metadata associated with the human subject; and
generate a report of the fall, wherein the generating is based on analyzing the second image sequence and the metadata.

17. The system of claim 16, further comprising the computing system storing the report in any combination of an electronic health records (EHR) database and an incident database.

18. The system of claim 16, wherein the metadata includes any combination of a patient history associated with the human subject, and environment information associated with an environment within which the human subject is present.

19. The system of claim 18, wherein the patient history includes a fall history associated with the human subject.

20. The system of claim 18, wherein the environment information includes a presence of any combination of a wheelchair, furniture, medical apparatus, obstacles, and a liquid spill.

21. The system of claim 18, wherein the environment information includes any combination of lighting, door states, window states, and a room temperature.

22. The system of claim 16, wherein the detecting of the fall is performed by at least one trained artificial intelligence (AI) system included in the computing system.

23. The system of claim 16, wherein the detecting of the fall further comprises the computing system performing an estimation of a pose and a gait of the human subject.

24. The system of claim 23, wherein the pose and gait estimation further comprises the computing system analyzing stability, balance, and movement patterns of the human subject preceding the fall.

25. The system of claim 16, wherein the detecting of the fall further comprises the computing system detecting a sudden change in a posture of the human subject, from a standing or a seated posture to a laying down position at a ground level.

26. The system of claim 16, wherein the caregiver is able to edit the report.

27. The system of claim 26, wherein an AI system included in the computing system uses the caregiver edits to update an AI model.

28. The system of claim 16, wherein the report is generated responsive to a request from the caregiver.

29. The system of claim 16, wherein each of the first image sequence and the second image sequence includes visual data of a respective and distinct time period before the fall and a respective and distinct time period after the fall.

30. The system of claim 16, wherein each of the first image sequence and the second image sequence is either of a video image sequence or a still image sequence.
